(19) United States
(12) Patent Application Publication
(10) Pub. No.: US 2006/0149284 A1
(43) Pub. Date: Jul. 6, 2006

(54) Insertion device and method for inserting a member within the body

(75) Inventors: William Cedrick McCormack, Corinth, MS (US); Craig M. Squires, Cordova, TN (US)

Correspondence Address: COATS & BENNETT, PLLC, P O BOX 5, RALEIGH, NC, 27602, US

(73) Assignee: SDGI Holdings, Inc.

(21) Appl. No.: 11/012,567

(22) Filed: Dec. 15, 2004

(51) Int. Cl. A61F 2/34

(52) U.S. Cl. 606/99

(57) ABSTRACT

A device for positioning and attaching a member within a patient. The device includes a handle and a connector. The handle comprises relatively movable first and second sections. The connector includes an alignment surface that may have an outwardly-extending mount. The connector further includes a pair of arms having an abutting surface and an inner surface. The device is movable between a first orientation with the alignment surface and the arms spaced apart, and a second orientation with the alignment surface and arms working in combination to hold the member in two separate directions.

### BACKGROUND

During a surgical procedure, it is often necessary to attach a member, such as a jig or implant, within a patient. Various devices are available for inserting and accurately positioning the member into a patient's body. However, the previous devices have various drawbacks.

Previous devices often do not apply the required holding force to maintain attachment with the member. The inadequate holding force could result in the member becoming detached during the insertion or removal from the patient. Detachment from the device could result in the member being improperly placed within the intervertebral space causing the physician further work to reconnect and correctly reposition the member.

In addition to not providing enough holding force, it often was difficult to attach the member to the device. The attachment locations on the members were minimal and required precise alignment with the device to attach the two together. If the alignment was incorrect, the two would not attach. Another difficultly was that the member would visibly appear to be securely attached to the device, even when the attachment was incorrect. The false attachment was not visibly obvious, and would only become apparent during the procedure when the member became detached at the improper time.

The difficulty of the attachment between the members and device further lead to a change in the construction of the members to facilitate attachment. The members would include additional mounting locations and structures. However, the additional locations and structure would deter from the functionality of the member to be used within the body, or did not ease the attachment process.

### SUMMARY

An embodiment of the present invention is directed to a device and method to position a member within the body. One specific embodiment features attachment of an implant within an intervertebral space between vertebral members. The device may include a handle that is manipulated during the procedure to insert and/or remove the member. The device may also include a connector mounted to the distal end of the handle for attaching the member. The device is selectively positionable between a first orientation that connects the member to the device, and a second orientation that releases the member such that it can remain within the body.

### BRIEF DESCRIPTION OF THE DRAWINGS

FIG. 1 is a perspective view of the device having a handle and a connector according to one embodiment of the present invention;

FIG. 2 is a cross-sectional view of the handle according to one embodiment of the present invention;

FIG. 3 is a side view of the member attached to the connector according to one embodiment of the present invention;

FIG. 4 is rear perspective view of the member attached to the device according to one embodiment of the present invention;

FIG. 5 is a perspective view of the member according to one embodiment of the present invention;

FIG. 6 is a top view of an initial stage of attaching the member to the device according to one embodiment of the present invention;

FIG. 7 is a top view of an intermediate stage of attaching the member to the device according to one embodiment of the present invention;

FIG. 8 is a top view of a final stage of attaching the member to the device according to one embodiment of the present invention; and

FIG. 9 is a partial cross-sectional view of one section of the handle according to another embodiment of the present invention.

### DETAILED DESCRIPTION

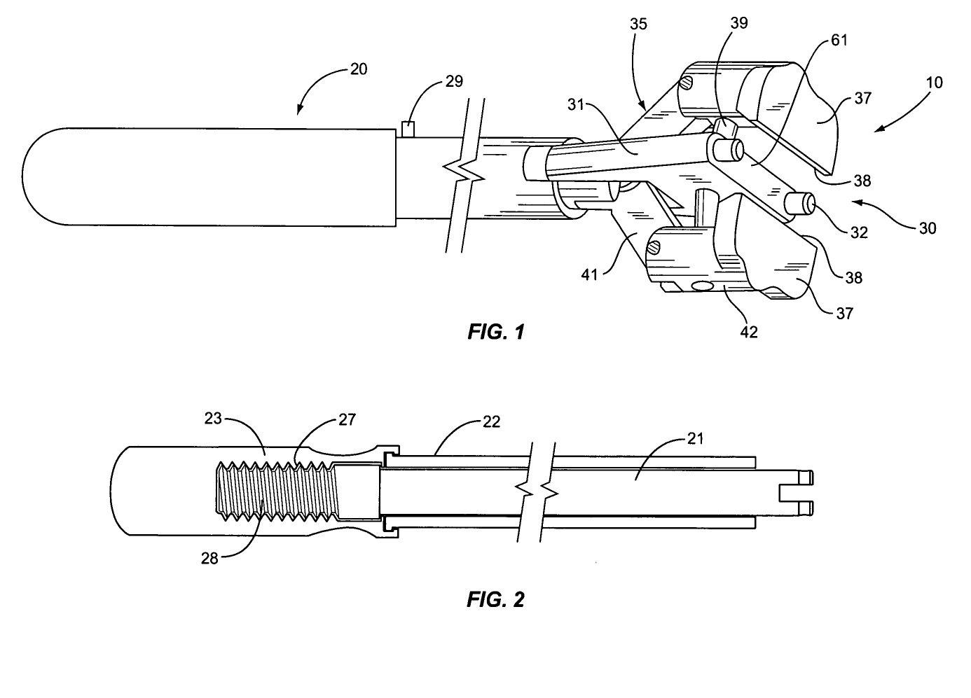

An embodiment of the present invention is directed to a device for positioning a member within the body of a patient. FIG. 1 illustrates the device, generally illustrated as 10, having a handle 20 and a connector 30. The device 10 is movable between a first orientation, as illustrated in FIG. 1, that releases the member 50 from the connector 20, and a second orientation that attaches the member 50 to the connector 20.

The handle 20 has a first end that attaches to the connector 20 and a second end that is manipulated by the physician during the procedure for attaching and releasing the member 50. The handle 20 includes an inner section 21 and an outer section 22. The sections 21, 22 are sized such that the two can move relative to each other. The length of the handle 20 may vary depending upon the application. In the embodiment illustrated in FIG. 2, the inner section 21 includes a distal end that attaches to the connector 30, and a threaded proximal end 28. The outer section 22 may extend around a portion or substantially the entirety of the inner section 21. A grip 23 is operatively connected to and rotatable relative to the outer section 22. The grip 23 includes a threaded section 27 that engages the threaded proximal end 28 of the inner section 21. The grip 23 may be rotated about the end of the outer section 22 to engage the inner section 21 causing the inner section 21 to axially move relative to the outer section 22. The inner section 21 is axially movable between first and second positions as will be explained in detail below.

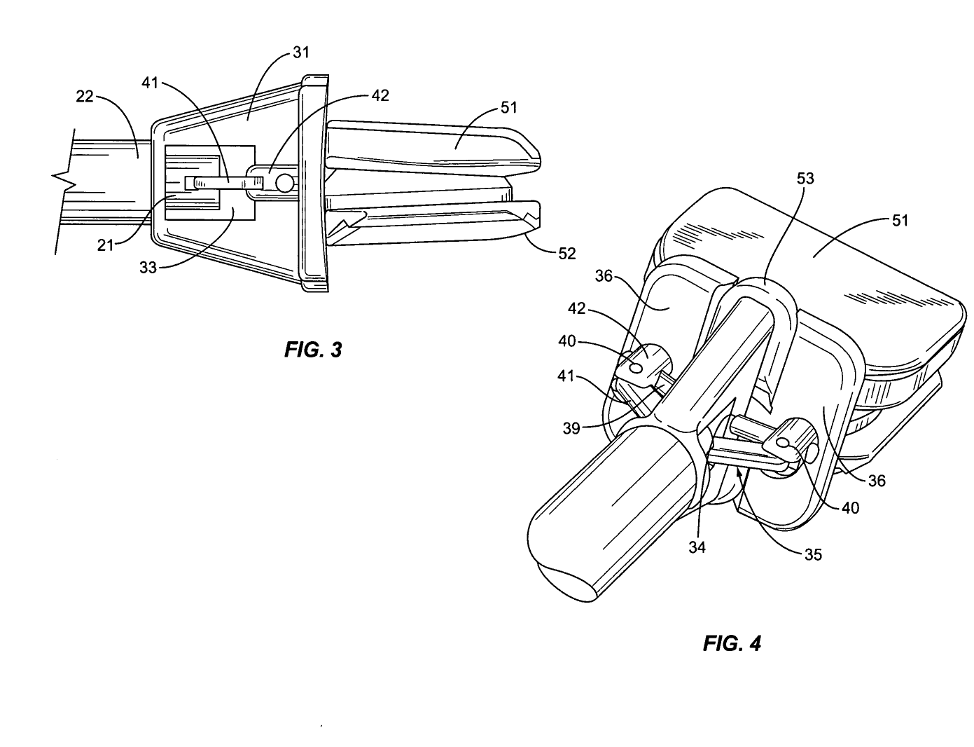

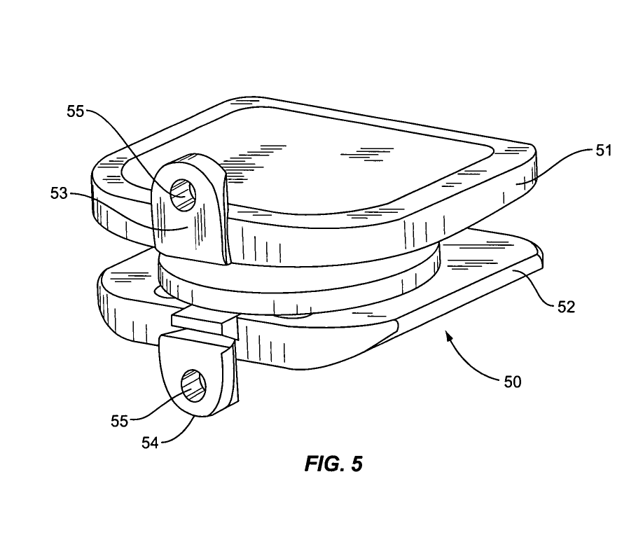

The connector 30 is attached to the distal end of the handle 20 and interacts with the member 50. The connector 30 comprises an extension 31, and a pair of arms 35 as illustrated in FIG. 1. The extension 31 extends axially outward beyond the distal end of the handle 20. Extension 31 is connected to the outer section 21 of the handle 20 and includes an alignment surface 61 and a pair of mounts 32 that attach to the member 50. The alignment surface 61 is substantially flat and contacts the member 50 during engagement. The mounts 32 have a substantially cylindrical shape and are spaced radially outward from a centerline of the handle with a first mount on a first side of the centerline and the second mount on a second side of the centerline. As illustrated in FIG. 3, an opening 33 may be positioned within a central section of the extension 31. As illustrated in FIG. 4, extension 32 may further include an aperture 34 for receiving guide arms 39.

The connector 30 further includes a pair of arms 32 connected to the inner section 21 of the handle 20. A first end of the arms 32 are pivotally connected together to the inner section 21. A pair of jaws 36 are positioned at the second end of the arms 32. The jaws 36 function in combination with the extension 31 to engage the member 50. The jaws 36 have an abutting surface 37 that abuts against the member 50, and a contact surface 38 that laterally contacts the member 50. In one embodiment, one or both of the abutting surface 37 and contact surface 38 are angled to conform to the shape of the member 50. In the embodiment best illustrated in FIG. 4, contact surface 38 has a curved edge that conforms to the shape and dimension of the member 50 to provide a secure attachment. The abutting surface 37 and contact surface 38, in combination with the alignment surface 61 of the extension 31 maintain the member 50 in both axial and lateral directions to provide a secure attachment.

Arms 35 may further include first and second sections 41, 42 connected together at a pivot 40. As illustrated in the embodiment of FIG. 4, the first section 41 may be connected to the handle 20, and the second section 42 includes the jaws 36.

Guide arms 39 may extend between the extension 31 and the arms 35. The guide arms 39 stabilize the arms 35 as they move between the first and second orientations. Guide arms 39 may have a variety of constructions, including a single member that extends through both arms 35 and through the extension 31, or a first guide arm that extends between the extension 31 and a first arm, and a second guide arm that extends between the extension 31 and a second arm. In one embodiment, the guide arm 39 extends through an aperture 34 in the extension. Arms 35 may further include apertures for receiving the outer ends of the guide arms 39.

The device 10 may be used on a variety of different members 50. One embodiment is best illustrated in FIG. 5 and includes upper and lower elements 51, 52, and mounting sections 53, 54. The upper and lower elements 51, 52 are be positioned within the intervertebral space, and the mounting sections 53, 54 are positioned outside of the intervertebral space, adjacent to the outer edges of the vertebral members. The mounting sections 53, 54 may include apertures 55 that receive the mounts 32 that extend outward from the extension 31. The apertures 55 may also be used to receive fasteners to attach the member 50 to the vertebral members.

The member 50 may comprise an articulating joint, such as the ball and trough arrangement as best illustrated in FIG. 5. Such ball and trough insert arrangements may include one of the first or second elements 51, 52 having a ball shape and the other having a corresponding hollow (or trough). Another embodiment is a “fusion” structure with the first and second elements 51, 52 each having an opening to expose the surface of the corresponding vertebral member. The elements 51, 52 are spaced apart a distance with an insert (not illustrated) mounted therebetween. The insert may comprise a portion of precision milled allograft bone harvested from a cadaver, a portion of autograft bone harvested from the same patient, or some synthetic material. One example of a commercially available product that may be used as a fusion-type insert is sold under the trademark “CORNERSTONE” by Medtronic Sofamor Danek of Memphis, Tenn. In one embodiment, the insert includes a central passage that aligns with the openings in the elements 51, 52. When the insert is positioned, the passage faces the apertures in the elements 51, 52, thereby providing a direct path from the vertebral members to the fusion insert, so as to promote osteoinduction and osteoconduction.

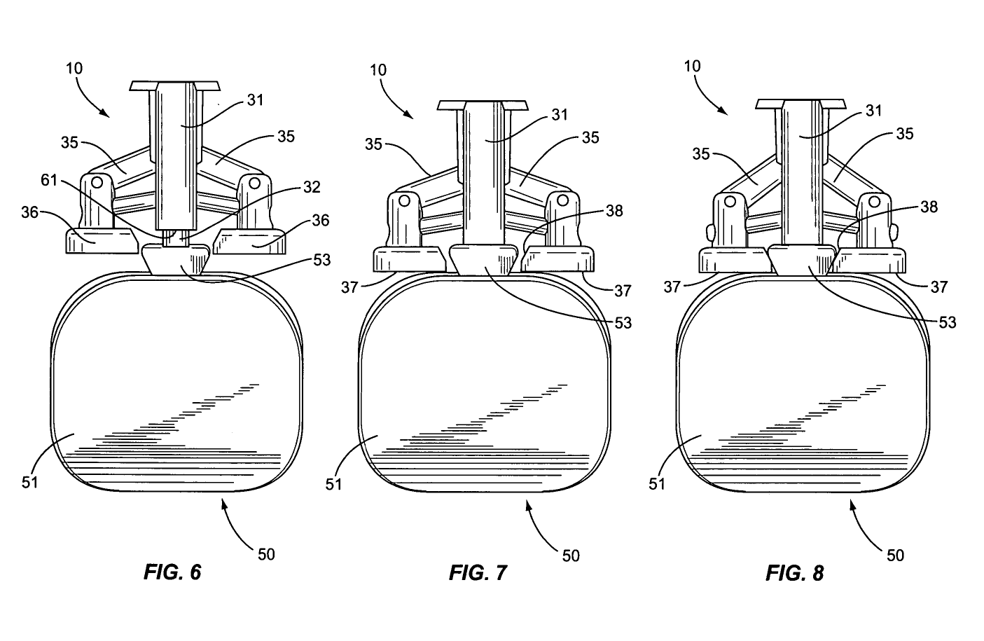

FIGS. 6, 7, and 8 illustrate the attachment of the device 10 to the member 50. As illustrated in FIG. 6, the alignment pins 32 that extend outward from the extension 31 are aligned with the apertures 55 in the mounting section 53 to align the device 10 with the member 50. FIG. 7 then illustrates the next step with the device 10 being moved axially farther forward with the alignment surface 61 contacting the surface of the first mounting section 53, and the abutting surface 37 contacting the edge of the first element 51. This movement further inserts the pin 32 into the aperture 55. FIG. 8 than illustrates the next step of moving the first section 21of the handle 20 axially backwards away from the member 50. This movement forces the jaws 36 to move inward with the contact surface 38 of the jaws 36 contacting against the lateral edges of the mounting section 53.

In the position illustrated in FIG. 8, the member 50 is securely attached to the device 10. The device 10 captures the member 50 in a lateral direction and in an axial direction. For the lateral direction, the contact surfaces 38 of the jaws 36 engage the edges of the mounting section 53. For the axial direction, the pin 32 is within the aperture 55 with the abutting surface 37 of the jaws against the edge of the member 50. Further axial support is provided by alignment surface 61 against the surface of the mounting section 53. FIGS. 6-8 illustrate a top view and it is understand that additional attachment support is being provided by the lower mount 32 and the interaction of the connector 30.

Once attached, the device 10 can be used to insert and position the member 50 into the intervertebral space. When the member 50 is in position, the procedure is reversed to detach the device 10. The first section 21 is moved forward causing the jaws 36 to move outward away from a centerline of the device 10. Once the contact surface 38 of the jaws 36 have moved beyond the mounting section 53, the device 10 can be axially moved away from the member 50.

Various embodiments are considered for the handle 20 to provide relative movement between the first and second sections 21, 22. The relative movement may result from one of the sections moving while the other remains substantially stationary, or movement of both sections. One embodiment of the handle 20 is illustrated in FIG. 2 and includes inner and outer sleeves 21, 22. Another embodiment of the handle 20 includes first and second members in a side-by-side orientation.

Further, the embodiments disclosed feature the extension 31 connected to the outer member 22, and the arms 35 connected to the inner member 21. The attachment may be reversed with the extension 31 connected to the inner member 21, and the arms 35 connected to the outer member 22. Further, the gripping section 23 in the embodiment of FIG. 2 provides the force for relative motion between the inner and outer members 21, 22. Other mechanisms may also be employed to provide the relative motion. One embodiment may feature a grip attached towards the proximal end of the handle 20 with a first grip section connected to the first section 21 and a second grip section 22 attached to the second section 22. Squeezing the grip sections causes relative movement between the sections 21, 22.

A lock 29 may also be positioned on the handle 20 to prevent relative motion of the first and second sections 21, 22. The lock 29 may be engaged at any point, and particularly when the device 10 is gripping the member 50 to prevent inadvertent detachment. In one embodiment, lock 29 is operatively connected to the gripping section 23 to prevent rotation. Another embodiment of the lock 29 includes a member that extends through the inner and outer sections 21, 22 in the locked position.

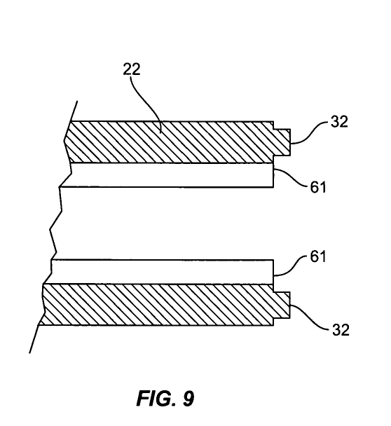

FIG. 9 illustrates another embodiment of the handle 20 that does not include an extension 31 mounted to the distal end. In this embodiment, the distal end of section 22 functions as the extension with the mounts 32 extending directly outward from the distal end, and the surface of the distal end itself functioning as the alignment surface 61. The other section 21 has not been illustrated, but is understood to include arms 35 that interact with the distal end of the section 22 such as that illustrated in FIG. 1.

While the illustrative embodiments discussed above have assumed that the member 50 replaces a single intervertebral disc, the present invention also encompasses situations where the member 50 replaces more than one intervertebral disc—a so-called corpectomy construct. This can be achieved through the use of a larger member 50, or by multiple members 50 that can be attached and detached from the device 10.

Additionally, although the devices and methods illustrated and described above are particularly useful in treating the cervical region of the spine, it should nevertheless be understood that the present invention is also applicable to other portions of the spine, including the lumbar or thoracic regions of the spine.

The term “vertebral member” and the like are used generally to describe the vertebral geometry comprising the vertebral body, pedicles, lamina, and processes. Likewise, the term “intervertebral space” and the like are used generally to describe the space between vertebral members. The intervertebral space may be formed between adjacent vertebral members, or between non-adjacent vertebral members. The member 50 may be sized and shaped, and have adequate strength requirements to be used within the different regions of the vertebra including the cervical, thoracic, and lumbar regions.

The term “member 50” is used herein in a general sense to describe a device that is selectively attached to the device 10. In one embodiment, the member 50 is an implant that is left in the body. In another embodiment, the member 50 is a jig which is a fixture or device to guide or hold a cutting, measuring, or space maintaining device in order to prepare a location, such as a vertebral member or intervertebral space, in order to receive an implant. Jigs may also be used in a process to relieve symptoms of a spinal or neurological disorder.

Other embodiments of the present invention may be carried out in other specific ways than those herein set forth without departing from the scope and essential characteristics of the invention. The terms “upper”, “lower”, “inner”, “outer”, and the like are terms to describe the relative positioning of different elements, and are used in a general sense. The present embodiments are, therefore, to be considered in all respects as illustrative and not restrictive, and all changes coming within the meaning and equivalency range of the appended claims are intended to be embraced therein.

What is claimed is:

1. A device to insert a member comprising:
a handle having first and second relatively movable sections; and
a connector mounted on a distal end of the handle, the connector comprising an axially-extending extension, and a pair of laterally-extending arms;
the extension attached to the first section and the pair of arms pivotally attached to the second section, with relative movement between the first section and the second section causing lateral movement of the arms between a first orientation positioned in proximity to the extension and a second orientation positioned away from the extension.

2. The device of claim 1, wherein the second section is positioned within the first section.

3. The device of claim 2, further comprising a gripping section attached to a proximal end of the handle and operatively connected to the first and second sections.

4. The device of claim 1, wherein the extension includes a pin that extends from the distal end of the extension and being axially aligned with the handle.

5. The device of claim 4, wherein the extension includes a second pin that extends from the distal end of the extension and being axially aligned with the handle.

6. The device of claim of claim 5, wherein the first pin is positioned radially outward from a centerline of the handle on a first side, and the second pin is positioned radially outward from the centerline of the handle on a second side opposite from the first side.

7. The device of claim 6, wherein the extension comprises a first leg mounted to a first side of the handle and a second leg mounted to a second side of the handle.

8. The device of claim 5, wherein each of the pins has a cylindrical shape.

9. The device of claim 1, wherein the pair of arms each includes a first end pivotally connected to the handle.

10. The device of claim 9, further comprising jaws pivotally connected to a second end of the pair of arms.

11. The device of claim 10, wherein the jaws comprise an angled inner edge that connected against the member when the arms are in the first orientation.

12. The device of claim 1, further comprising a lock operatively connected to the handle to prevent relative movement between the first section and the second section.

13. The device of claim 1, further comprising a pair of guide arms extending between the extension and the pair of arms to guide the pair of arms between the first and second orientations.

14. The device of claim 1, wherein the extension is positioned between the pair of arms.

15. A device to insert a member comprising:
a handle having a first section and a second section, the sections being relatively movable between first and second orientations;
an extension mounted to the first section and having a substantially flat mounting surface to contact the member and a mount extending outward from the mounting surface; and
arms each having a first end connected to the second section and a second end spaced from the first end to be aligned with the mounting surface, the second ends being in lateral proximity to the mounting surface in the first orientation, and the second ends being laterally spaced away from the mounting surface in the second orientation.

16. The device of claim 15, wherein the arms comprise first and second portions mounted together at an intermediate joint.

17. The device of claim 15, further comprising guide arms extending between the extension and the arms to guide the arms between the first and second orientations, the guide arms positioned between a first end of the arms and the mount.

18. A device to insert a member comprising:
a handle having a first section and a second section, the sections being relatively movable between first and second orientations;
an extension mounted to the first section and having a substantially flat mounting surface to contact the member; and
arms each having a first end connected to the second section and a second end having an abutting surface and inner edges, in the first orientation the abutting surface being aligned in a first plane and the mounting surface aligning in a second plane and the inner edges being laterally spaced away from the mounting surface, and in the second orientation the abutting surface being aligned in the first plane and the mounting surface being aligned in a third plane axially outward a greater distance than the second plane and the inner edges being in lateral proximity to the mounting surface.

19. The device of claim 18, wherein the first plane, second plane, and third plane are all in a substantially parallel alignment.

20. A device to insert a member comprising:
a handle having relatively movable first and second sections;
a substantially flat mounting surface positioned on the distal end of the first section to contact the member;
arms having a first end connected to the second section and a second end positioned outside of the mounting surface, the arms movable between a first orientation with the second ends in proximity to the mounting surface to contact the member, and a second orientation with the second ends spaced away from the mounting surface and the member.

21. The device of claim 20, further comprising a cylindrical mount extending outward from the mounting surface to engage the member.

22. A device to position a member within a human body comprising:
a handle having a first section and a second section;
an extension mounted to the second section and having a mounting surface on a distal end and a mount extending outward from the mounting surface to engage the member;
arms having first ends mounted to the first section and second ends that extend outward beyond the handle; and
the first and second sections being relatively movable to laterally position the arms between a first orientation with the second ends adjacent to the mount and a second orientation with the second ends spaced away from the mount.

23. The device of claim 22, wherein the first section is positioned on an inside of the second section.

24. The device of claim 23, further comprising a actuating mechanism to selectively move the first section relative to the second section.

25. The device of claim 22, wherein the first ends of the arms are mounted within the extension.

26. The device of claim 22, further comprising guide arms extending between the extension and the arms to guide the second ends between the first and second orientations.

27. A device to position a member within an intervertebral space comprising:
a handle comprising a first section and a second section;
a pair of mounts extending outward from a distal end of the handle;
a first jointed arm positioned on a first side of the handle, the first jointed arm comprising a first inner member jointedly mounted to a first outer member; and
a second jointed arm positioned on a second side of the handle, the second jointed arm comprising a second inner member jointedly mounted to a second outer member;
the first and second sections being relatively movable between a first orientation with the first and second outer members in proximity to the pair of mounts and a second orientation with the first and second outer member laterally spaced away from the pair of mounts.

28. The device of claim 27, wherein the first and second sections are nested together.

29. The device of claim 27, further comprising an actuating mechanism to cause relative movement between the first section and the second section.

30. The device of claim 27, further comprising a lock operatively connected to the first and second sections to prevent relative motion between the first and second sections.

31. The device of claim 27, wherein each of the first and second sections are elongated rods.

32. The device of claim 27, further comprising an extension mounted to the distal end of the first section, the extension having a flat surface from which the pair of mounts extend.

33. The device of claim 32, further comprising a guide arm extending between the extension and the first outer member of the first jointed arm, the guide arm guiding the movement of the first jointed arm between the first and second orientations.

34. The device of claim 33, further comprising a second guide arm extending between the extension and the second outer member of the second jointed arm, the guide arm guiding the movement of the second jointed arm between the first and second orientations.

35. The device of claim 34, wherein the guide arm and the second guide arm are a unitary piece.

36. The device of claim 27, wherein the first and second outer members each comprise a substantially flat abutting surface that contacts the member in the first orientation.

37. The device of claim 27, wherein the first and second outer members each comprise an angled contact surface on an inner edge that contacts the member in the first orientation.

38. The device of claim 27, wherein the pair of mounts are each cylindrical and spaced a distance apart.

39. A device to position a member within an intervertebral space comprising:
a handle comprising relatively movable inner and outer sections;
a planar mounting surface positioned at a distal end of one of the inner and outer sections;
a first jointed arm positioned on a first side of the handle, the first jointed arm comprising a first inner member jointedly mounted to a first outer member;
a second jointed arm positioned on a second side of the handle, the second jointed arm comprising a second inner member jointedly mounted to a second outer member;
the first and second jointed arms being connected to a second of the inner and outer sections, the inner and outer sections relatively movable between a first orientation with the first and second outer members in proximity to the handle and a second orientation with the first and second outer member laterally spaced away from the handle.

40. The device of claim 39, further comprising a mount extending outward from the mounting surface to engage the member.

41. The device of claim 40, further comprising a second mount extending outward from the mounting surface, the second mount spaced apart from the mount.

42. A device to position a member comprising:
an elongated handle having a first section and a second section;
a planar alignment surface positioned at a distal end of the first section;
a pair of arms each having a first end connected to the second section and a second end extending outward therefrom and having a contact surface on an inner edge, the pair of arms being laterally movable between an open orientation with the contact surfaces spaced from the alignment surface and a closed orientation with the contact surfaces in proximity to the alignment surface, in the open orientation the member is in contact with only the alignment surface, and in the closed orientation the member is contacted by the alignment surface and both of the inner contact surfaces.

43. The device of claim 42, wherein the handle comprises first and second sections with the alignment surface positioned on the first section and the pair of arms connected to the second section.

44. The device of claim 42, wherein the first ends of the pair of arms are pivotally connected to the handle, and each of said pair of arms comprises an intermediate pivot point between the first and second ends.

45. The device of claim 42, wherein the enlarged alignment surface is substantially flat and lies in a plane substantially perpendicular to a centerline of the first section.

46. A device to position a member comprising:
an elongated handle having a first section and a second section;
an enlarged alignment surface positioned at a distal end of the first section, the alignment surface being substantially flat and lies in a plane substantially perpendicular to a centerline of the first section.
a pair of arms each having a first end connected to the second section and a second end extending outward therefrom and having a contact surface on an inner edge, the pair of arms being laterally movable between an open orientation with the contact surfaces spaced from the alignment surface and a closed orientation with the contact surfaces in proximity to the alignment surface.

47. A device to position a member comprising:
an elongated handle having a first section and a second section, the first and second sections being relatively movable between a first orientation and a second orientation;
an alignment surface at a distal end of the first section;
a pair of arms positioned on outer sides of the alignment surface, each pair of arms having a first end connected to the second section and a second end extending outward therefrom and having a contact surface on an inner edge;
the contact surfaces being spaced from the alignment surface in the first orientation such that the member is in contact with the alignment surface, and the contact surfaces being in proximity to the alignment surface in the second orientation such that the member is in contact with the alignment surface and the contact surfaces.

48. A device to position a member comprising:
an elongated handle having a first section and a second section;
a planar alignment surface positioned at a distal end of the first section;
a pair of arms each having a first end connected to the second section and a second end extending outward therefrom and having a contact surface on an inner edge, the pair of arms being laterally movable between an open orientation with the contact surfaces spaced from the alignment surface and a closed orientation with the contact surfaces in proximity to the alignment surface, in the open orientation the member is in contact with only the alignment surface, and in the closed orientation the member is contacted by the alignment surface and both of the inner contact surfaces.

49. The device of claim 48, wherein the handle comprises first and second sections with the alignment surface positioned on the first section and the pair of arms connected to the second section.

50. The device of claim 48, wherein the first ends of the pair of arms are pivotally connected to the handle, and each of said pair of arms comprises an intermediate pivot point between the first and second ends.

51. The device of claim 48, wherein the enlarged alignment surface is substantially flat and lies in a plane substantially perpendicular to a centerline of the first section.

52. A device to position a member comprising:
an elongated handle having a first section and a second section;
an enlarged alignment surface positioned at a distal end of the first section, the alignment surface being substantially flat and lies in a plane substantially perpendicular to a centerline of the first section.
a pair of arms each having a first end connected to the second section and a second end extending outward therefrom and having a contact surface on an inner edge, the pair of arms being laterally movable between an open orientation with the contact surfaces spaced from the alignment surface and a closed orientation with the contact surfaces in proximity to the alignment surface.

53. A method of attaching a member to a device, the method comprising the steps of:
engaging a first surface on a distal end of a first handle section with the member;
axially moving a proximal section of the first handle section relative to a proximal section of a second handle section; and
laterally engaging a pair of arms on a distal end of the second handle section with the member.

54. The method of claim 53, wherein the step of axially moving the proximal section of the first handle section relative to the proximal section of the second handle section comprises moving the first handle section within the second handle section.

55. A method of attaching a member to a device, the method comprising the steps of:
extending a distal section of a first handle outward from a distal section of a second handle;
engaging a contact surface on the distal section of the first handle with the member;
axially moving the second handle relative to the first handle causing a pair of arms on the distal section of the second handle to laterally move inward and contact the member.
